SEQUENCE LISTING (1 sequence)

SEQ ID NO: 1 - DNA, 18 bp, synthetic construct
  source 1..18: mol_type = other DNA
ccctaaccctaaccctaa

(19) United States
(12) Patent Application Publication
(10) Pub. No.: US 2024/0050534 A1
(43) Pub. Date: Feb. 15, 2024

(54) USE OF COLLAGEN COMPOSITIONS FOR INCREASING TELOMERE LENGTH

(71) Applicant: BioCell Technology, LLC, Irvine, CA (US)

(72) Inventor: Suhail Ishaq, Villa Park, CA (US)

(21) Appl. No.: 18/096,941

(22) Filed: Jan. 13, 2023

Related U.S. Application Data
(63) Continuation of application No. 17/349,669, filed on Jun. 16, 2021, now abandoned.

(51) Int. Cl. A61K 38/39; A61K 31/196

(52) U.S. Cl. CPC A61K 38/39; A61K 31/196

(57) ABSTRACT

The present invention relates to use of a collagen composition for increasing telomere length in a cell, for treating or preventing conditions associated with reduced/shortened telomere length, and for dietary supplement and functional food uses for promoting healthy aging and longevity by mitigating telomere shortening.

### FIELD OF THE INVENTION

The present invention relates to use of a collagen composition for increasing telomere length in a cell, for treating or preventing conditions associated with reduced/shortened telomere length, and for dietary supplement and functional food uses for promoting healthy aging, longevity, and wellness by mitigating telomere shortening.

### INCORPORATION BY REFERENCE OF THE SEQUENCE LISTING

This application contains a Sequence Listing that has been submitted in .xml format via EFS-Web and is hereby incorporated by reference in its entirety. The copy was created on Nov. 3, 2023, is named “SequenceListing_078479_1000_02USCN,” and is 2 kilobytes in size.

### BACKGROUND OF THE INVENTION

Telomeres are DNA sequences at the tips of chromosomes, with a structure that protects chromosomes from eroding. In every cell division during mitosis, some genetic material at the end of chromosomes is lost. Telomeres ameliorate the problem by providing a repetitive template for enzymatic repair of chromosome ends. Telomere repair is mediated by the enzyme telomerase. Short telomere length is known to arrest cell proliferation, and to cause senescence and apoptosis. Telomere length also limits the number of times cells can divide, known as the Hayflick limit. Transfecting cells with the telomerase gene can allow them to proliferate indefinitely. Telomere shortening is associated with a number of medical conditions, including bone marrow failure, dyskeratosis congenita, acquired aplastic anemia, pulmonary fibrosis, and liver disease, among others.

Accordingly, there is a need in the art for improved methods to increase telomere length in cells and treat conditions associated with reduced telomere length.

### SUMMARY OF THE INVENTION

Provided herein is a method of increasing telomerase activity, increasing average telomere length, reducing the rate of telomere shortening, and/or increasing proliferative capacity of a cell. The method may comprise contacting the cell with a composition comprising hydrolyzed collagen. The cell may be an epithelial cell, epidermal cell, myocardial cell, bone marrow cell, B lymphocyte, T lymphocyte, embryonic stem cell, leukocyte, fibroblast, chondrocyte, or osteoblast. In particular, the cell may be a fibroblast. The cell may be from a subject suffering from a condition associated with reduced telomere length.

Further provided herein is a method of treating or preventing a condition associated with reduced telomere length. The method may comprise administering a composition to a subject in need thereof. The condition may be bone marrow failure; hematopoietic dysfunction; aplastic anemia (which may be acquired); lung fibrosis; liver fibrosis; cancer; inflammatory bowel disease; ulcerative colitis; Barrett's esophagus; cardiovascular disease; a degenerative disease associated with aging; a telomere syndrome, dyskeratosis congenital; Hoyeraal-Hreidarsson syndrome; Revesz syndrome; cerebroretinal microangiopathy; Fanconi anemia; cryptogenic cirrhosis; nodular regenerative hyperplasia; Rothmund-Thomson syndrome; Immunodeficiency, centromeric region instability, and facial anomalies type I; or, fascioscapulohumeral muscular dystrophy. The cancer may be colorectal, esophageal, a hematologic cancer, head and neck squamous-cell carcinoma, skin or anorectal cancer, myelodysplastic syndrome, leukemia, or acute myeloid leukemia.

Also provided is a method or promoting healthy aging, longevity, and/or wellness. The method may comprise administering a composition to a subject in need thereof.

The composition may comprise hydrolyzed collagen. The hydrolyzed collagen may have an average molecular weight of between about 50 and about 10,000 Da and at least about 20% glycosaminoglycans. The hydrolyzed collagen may comprise type II collagen. The composition may comprise at least about 60% protein. The composition may comprise at least about 60% hydrolyzed type II collagen.

The composition may comprise at least about 15% chondroitin sulfate. The composition may comprise at least about 5% hyaluronic acid. The composition may comprise at least 60% hydrolyzed type II collagen, 20% chondroitin sulfate, and 10% hyaluronic acid. The composition may be or may comprise a hydrolyzed chicken sternal cartilage extract.

For use with cells, the composition may be at a concentration 25-1000 μg/mL. The composition may comprise 25-1000 μg/mL of the hydrolyzed chicken sternal cartilage extract. The composition may be contained in or combined with a cell culture medium. For treating or preventing conditions, or for promoting healthy aging, longevity, and/or wellness, the composition may be an oral formulation or a topical formulation.

### BRIEF DESCRIPTION OF THE DRAWINGS

FIG. 1 shows a standard curve of Ct values of protein from HeLa cells against log of protein (ng). The cycle number at the threshold (Ct value) for each sample is interpolated in the curve, in order to calculate the relative telomerase activity (RTA).

FIG. 2 shows telomerase activity for different amounts of a collagen composition described herein (referred to as “BCC”). Graph bars of Q-TRAP results (RTA) for in vitro treatments in cultures of primary Fibroblasts. Data were grouped by condition and time point of treatment.

FIG. 3 shows growth curves of human adult primary fibroblasts untreated (control) and treated with the BCC (250, 83.3 and 27.8) under standard conditions. Each point on the population curve represents the average of the triplicates for each cell passage.

FIG. 4 shows growth curves of human adult primary fibroblasts untreated (control-DMEM) and treated with BCC (250, 83.3 and 27.8) under oxidative conditions (10 μM H₂O₂). Each point on the population curve represents the average of triplicates average for each cell passage.

FIG. 5A-B are histograms showing the distribution of telomere lengths in representative samples. Bars represent the relative frequency for every particular fluorescence intensity normalized (x-axis). The 20th percentile (darker bars) indicates the particular length below which 20% of the telomeres have been observed. The median (MTL) and average (ATL) telomere length are also indicated in the histogram. This histogram also allows for the analysis of telomere length variability.

FIG. 6 shows both the DAPI stained nuclei and the fluorescently labeled telomeric spots (white spots) inside each nucleus of a representative TAT image.

FIG. 7A-C show bar charts of TAT results for different time points and treatments under standard conditions. FIG. 7A. Median Telomere Length. FIG. 7B. 20th Percentile Telomere Length. FIG. 7C. % Short Telomeres.

FIG. 8A-C show bar charts of TAT results for different time points and treatments under oxidative conditions. FIG. 8A. Median Telomere Length. FIG. 8B. 20th Percentile Telomere Length. FIG. 8C. % Short Telomeres.

FIG. 9 shows bar charts of shortening rate for different time points and treatments under standard conditions.

FIG. 10 shows bar charts of shortening rate for different time points and treatments under oxidative conditions.

### DETAILED DESCRIPTION

The inventors have discovered that, surprisingly, the collagen compositions disclosed herein increase telomerase activity, increase average telomere length, and reduce the rate of telomere shortening in cells, and can be used to treat or prevent conditions associated with reduced telomere length.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

“Treatment” or “treating,” when referring to protection of an animal from a disease, means suppressing, repressing, reducing, or completely eliminating the disease. Suppressing the disease involves administering a composition of the present invention to an animal after induction of the disease but before its clinical appearance. Repressing the disease involves administering a composition of the present invention to an animal after clinical appearance of the disease. “Preventing” the disease involves administering a composition of the present invention to an animal prior to onset of the disease.

### 2. Collagen Composition

Provided herein is a composition comprising collagen. The average molecular weight of the collagen may be between about 50 and 10,000 Daltons, between about 1,500 and 2,500 Daltons, and in particular may be about 5,500 Daltons. The collagen may comprise collagen peptides. The collagen may be hydrolyzed, and may be type II collagen. As measured on a dry weight basis, the collagen composition may comprise at least about 60% hydrolyzed type II collagen, and may comprise at least about 75% hydrolyzed type II collagen. The amount of collagen may be calculated as Total Protein by assay method, which may be AOAC 992.15/Ph.Eur., which is a standard known in the art. The collagen composition may comprise at least 60% protein, as measured on a dry weight basis. The moisture content of the collagen composition may be between 5% and 7%.

The composition may also comprise one or more glycosaminoglycans, particularly at least one of chondroitin sulfate, hyaluronic acid, and other proteoglycans. The collagen composition may comprise, on a dry weight basis, at least about 20% glycosaminoglycans. In one example, the collagen composition comprises at least about 15% or 20% chondroitin sulfate. The amount of chondroitin sulfate may be calculated by assay method USP CPC Titration or Enzymatic Hydrolysis HPLC, which is known in the art. The chondroitin sulfate may be depolymerized. The composition may also comprise at least about 5% or 10% hyaluronic acid, as measured on a dry weight basis. The amount may be calculated by assay method Euro|Pharma UV-Vis, which is known in the art. In one example, the collagen composition comprises at least about 15% chondroitin sulfate and at least about 5% hyaluronic acid. In another example, the collagen composition contains at least about 60% hydrolyzed type II collagen, 20% chondroitin sulfate, and 10% hyaluronic acid.

The amino acid composition of an exemplary collagen composition is shown in Table 1.

TABLE 1
Amino acid composition of hydrolyzed collagen type II

| Amino acid | g/100 g product |
|---|---|
| arginine | 4.42 |
| histidine | 2.05 |
| isoleucine | 1.90 |
| leucine | 4.20 |
| lysine | 3.54 |
| methionine | 1.38 |
| phenylalanine | 2.14 |
| threonine | 2.60 |
| tryptophan | 0.37 |
| alanine | 4.51 |
| asparagine/aspartic acid | 5.29 |
| cystine | 0.46 |
| glutamine/glutamic acid | 8.75 |
| glycine | 8.93 |
| hydroxyproline | 3.90 |
| proline | 5.25 |
| serine | 2.45 |
| tyrosine | 1.16 |
| valine | 2.43 |

The specifications of an exemplary collagen composition are shown in Table 2.

TABLE 2
Test results of collagen composition

Specification

Typical analysis

| Purity | 100% | |
|---|---|---|
| Solubility | Water-Soluble | |
| Chondroitin Sulfate | ~20.0% | |
| Hyaluronic Acid | ~10.0% | |
| Other Proteoglycans | ~3.0% | |
| Collagen Type II Protein | ~62.7% | |
| Nitrogen | 12.1% | |
| Fat | 0.0% | |
| Loss on Drying | <8% | |
| Heavy Metals | <5 | ppm |

| | |
|---|---|
| Ash | 8.3% |
| Calcium | 530 mg/100 gms |
| Magnesium | 161 mg/100 gms |
| Potassium | 1961 mg/100 gms |

Microbiological Analysis

| | | |
|---|---|---|
| Standard Plate Count | <1000 | gms |

| | |
|---|---|
| E. Coli | Negative |
| Salmonella | Negative |

Physical Analysis

| Appearance | Off white/yellow fine powder | |
|---|---|---|
| Odor | Characteristic | |
| Particle Size | >100 | mesh |

Exemplary collagen compositions have the following specifications.

TABLE 3

| | SPECIFI- | TYPICAL | |
|---|---|---|---|
| ANALYSIS | CATIONS | RESULTS | METHOD |
| APPEARANCE: | Fine Powder | Fine Powder | Organoleptic |
| COLOR: | Off white to | Off white to | Organoleptic |
| | beige | beige | |
| ODOR: | Characteristic | Characteristic | Organoleptic |
| SOLUBILITY: | Water Soluble | Water Soluble | 1% in H2O |
| CHONDROITIN | Not less than | 20%* | USP CPC |
| SULFATE (On | (NLT) 20.0% | | Titration |
| Dry Basis): | | | |
| HYALURONIC | NLT 10.0% | 10%* | Euro Pharma |
| ACID (On Dry | | | UV-Vis |
| Basis): | | | |
| HYDROLYZED | NLT 60.0% | 75%* | AOAC 992.15/Ph. |
| COLLAGEN | | | Eur. 2.5.9 |
| TYPE II AS | | | |
| TOTAL | | | |
| PROTEIN (On | | | |
| Dry Basis): | | | |
| ASH: | Not more than | Conforms | AOAC 942.05/Ph. |
| | (NMT) 12.0% | | Eur. 2.4.16 |
| FAT: | NMT 2.0% | Conforms | AOAC 922.06 |
| LOSS ON | NMT 5.0% | Conforms | AOAC 930.15/Ph. |
| DRYING: | | | Eur. 2.2.32 |
| HEAVY | NMT 5.0 PPM | Conforms | AOAC |
| METALS: | | | 2013.06/USP<730> |
| Lead | NMT 0.2 PPM | Conforms | AOAC |
| | | | 2013.06/USP<730> |
| Cadmium | NMT 0.1 PPM | Conforms | AOAC |
| | | | 2013.06/USP<730> |
| Mercury | NMT 0.1 PPM | Conforms | AOAC |
| | | | 2013.06/USP<730> |
| Arsenic | NMT 1.0 PPM | Conforms | AOAC |
| | | | 2013.06/USP<730> |
| BULK DENSITY | 0.40-0.60 g/cc | Conforms | USP<616>/Ph. Eur. |
| (Untapped): | | | 2.2.42 |
| PARTICLE | 100% through | Conforms | USP<786>/Ph. Eur. |
| SIZE: | 80 mesh | | 2.9.12 |
| AVERAGE | 1,500-2,500 | Conforms | HPLC |
| MOLECULAR | Daltons | | |
| WEIGHT: | | | |

TABLE 4

| | | | |
|---|---|---|---|
| STANDARD | NMT | Conforms | AOAC Petrifilm/Ph. |
| PLATE COUNT | 10,000 | | Eur. 5.1.4 |
| | cfu/g | | |
| YEAST & MOLD | NMT 500 | Conforms | AOAC Petrifilm/Ph. |
| | cfu/g | | Eur. 5.1.4 |
| E. COLI | NEGATIVE | Negative/10 | USP<2022>/Ph. |
| | | g | Eur. 5.1.4 |
| SALMONELLA | NEGATIVE | Negative/25 | USP<2022>/Ph. |
| | | g | Eur. 5.1.4 |
| STAPHYLOCOCCUS | NEGATIVE | Negative/10 | USP<2022>/Ph. |
| AUREUS | | g | Eur. 5.1.4 |

The collagen composition may be as described in any one or more of U.S. Pat. Nos. 6,780,841; 7,091,180; 7,799,348; 6,025,327; 6,323,319; and 8,563,045, the contents of which are incorporated herein by reference in their entirety.

The collagen composition may be hydrolyzed chicken cartilage extract. The cartilage may be extracted from the sternum, and the collagen composition may be 100% pure hydrolyzed chicken sternal cartilage extract. The sternum cartilage may be cut from the chicken carcass, then subjected to an aqueous enzyme assisted extraction technique using food-grade proteases. This process may produce a hydrolysate that is then sterilized, filtered, concentrated under vacuum, and spray dried to form the finished powder.

In one example, the collagen composition is derived from the extracellular matrix of food-grade animal tissue, including gelatin production. In another example, the collagen composition is prepared by a fermentation-based process method.

The collagen composition may be prepared by a method described in U.S. Pat. No. 7,091,180, the contents of which are incorporated herein by reference. The method may comprise cutting fresh sternal cartilage from chicken carcasses and removing all meat therefrom. The sternal cartilage may be cut, leaving a space of about two millimeters from the bone to avoid removing any bone fragments. The fresh sternal cartilage may then be promptly frozen and the remains of the chicken carcass are discarded. The sternal cartilage may be used exclusively for preparing a collagen type II powder. The chicken sternal cartilage may be processed according to good manufacturing procedures (GMP). Other contemplated sources of collagen type II are mammalian cartilage (e.g. bovine, ovine, capra, porcine or avian) and marine sources (e.g. jelly fish or shark fins).

In one example, whole cartilage is suspended in an aqueous solution, which may be water, for about one hour at about 35° C. at a pH of between about 4 and 8. The pH may in particular be between about 6 and 7. In another example, the pH is about 6.5. The water is removed, and the cartilage is incubated with one or more proteases obtainable from a natural source (i.e. papain, ficin, bromelain) for between about 2 and 10 hours, particularly about 6 hours, at about 35° C.-55° C. at a pH of between about 4 and 8 to form a hydrolysate. The pH will depend on the pH optimum of the particular enzyme(s) used for the hydrolysis and are well known to one of ordinary skill in the art. The hydrolysate is then sterilized for about 30 minutes at a temperature between about 95° C. and 105° C. The sterilized hydrolysate is filtered through diatomaceous earth, concentrated, which may be under vacuum, dried to form a powder and packed. Other filtration methods are contemplated, including vacuum filtration. The collagen composition may be spray dried using a size 56 pressure nozzle into a heat tunnel. The final particle size and mesh are adjusted to 0.46 g/cc, yielding a fine powder. The powder is packed in a 40 kg drum with a plastic bag liner. The powder is water soluble.

For use on cells, such as in cell culture, the collagen composition may be contacted with the cell at a concentration of about 27.8 to 250 μg/mL. In particular, the concentration may be about 25, 27.8, 50, 75, 83.3, 100, 125, 150, 175, 200, 225, 250, 300, 350, 400, 450, 500, 550, 600, 700, 750, 800, 850, 900, 950, or 1000 μg/mL, or within a range of two of the foregoing values. The collagen composition may be a supplement to or contained in a cell growth or cell culture medium. Such media are well known in the art. The collagen composition may also be used in a buffering solution.

The collagen composition may be an oral formulation or nutritional supplement. The composition may be a dispersible powder (e.g., in a sachet), aqueous solution, granule, pellet, tablet, aqueous or oil suspension, emulsion, hard or soft capsule, soft gel, gummy, chew, extruder bar, syrup, or elixir. Compositions intended for oral use may be prepared according to any method known in the art for the manufacture of pharmaceutical or dietary compositions and such compositions may contain one or more of the following agents: sweeteners, flavoring agents, coloring agents and preservatives. The sweetening and flavoring agents will increase the palatability of preparation.

The oral formulation may be a tablet, which may contain the collagen composition in admixture with non-toxic pharmaceutically acceptable excipients suitable for tablet manufacture. Such excipients include inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. Tablets may be uncoated or may be coated by known techniques to delay disintegration and absorption in the gastrointestinal tract and thereby provide a sustained action over a longer period of time. For example, a time delay material such as glyceryl monostearate or glyceryl distearate alone or with a wax may be employed. The use of enteric coatings is also contemplated.

The oral formulation may also be a hard gelatin capsule, which may comprise the collagen composition mixed with an inert solid diluent, for example calcium carbonate, calcium phosphate or kaolin. The oral formulation may also be a soft gelatin capsule, which may comprise the collagen composition mixed with water or an oil medium, such as peanut oil, liquid paraffin or olive oil.

The oral formulation may be an aqueous suspension, which may contain the collagen composition in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin. The oral formulation may also be an oil suspension, which may be formulated by suspending the active ingredient in a vegetable oil, such as arachis oil, olive oil, sesame oil or coconut oil, or in a mineral oil such as liquid paraffin. The oil suspension may contain a thickening agent, such as beeswax, hard paraffin or cetyl alcohol. Sweetening agents, such as those set forth above, and flavoring agents may be added to provide a palatable oral preparation. These compositions may be preserved by an added antioxidant such as ascorbic acid. Dispersible powders and granules of the invention suitable for preparation of an aqueous suspension by the addition of water provide the active ingredient in admixture with a dispersing or wetting agent, a suspending agent, and one or more preservatives. Additional excipients, for example sweetening, flavoring and coloring agents, may also be present.

The oral formulation may be a syrup or elixir, which may comprise the collagen composition formulated with one or more sweetening agents, such as glycerol, sorbitol or sucrose. Such formulations may also contain a demulcent, a preservative, a flavoring agent and/or a coloring agent.

The collagen composition may be mixed with other ingestible forms and consumed in solid, semi-solid solution, suspension or emulsion form. It may also be mixed in conjunction or alternatively with pharmaceutically acceptable carriers, flavor enhancers, water, suspending agents and emulsifying agents. The collagen composition may also be provided in tablet form in admixture with ascorbic acid.

The collagen composition may be incorporated into a nutraceutical product or dietary supplement. Use of the collagen composition in a nutraceutical product or dietary supplement may be appropriate for a healthy subject, or a subject who has a condition described herein. In one example, the collagen composition is incorporated into food or drink as an additive. The collagen composition may be incorporated into a food product, which may be a tea-based beverage, juice, soft drink, coffee, milk, jelly, cookie, cereal, chocolate, snack bar, herbal extract, or dairy product (e.g., ice cream or yogurt). In one example, the collagen composition is mixed with a citrus juice such as orange, grapefruit or tangerine due to the promotion of connective tissue formation by ascorbic acid.

The nutraceutical product may be used to supplement the diet. The nutraceutical product may comprise one or more isolated nutrients and herbal products in addition to the collagen composition. The nutraceutical composition may comprise one or more of a vitamin, a mineral, an herb or other botanical, an amino acid, a metabolite, constituent, and an extract. The nutraceutical product may comprise one or more of vitamin C, silicon, and silica.

In another example, the collagen composition is formulated in a topical formulation. The topical formulation may be a cream, serum, or lotion.

The collagen composition may be administered in a daily dosage of between about 25 mg and 500 mg; between about 500 mg and 5,000 mg; between about 2,000 mg and 4,000 mg; between about 5,000 mg and 15,000 mg; or between about 1,000 and 3,000 mg. The daily dosage of the collagen composition may be 500, 1000, or 1200 mg.

In one example, the collagen composition is formulated into a tablet, which may comprise the collagen composition at a dose of about 300 mg to 1,000 mg per tablet. Each tablet may comprise 1,200 mg of the collagen composition. In one example, the collagen composition is formulated into 500 mg tablets and 4-6 tablets are taken daily. The tablets may be taken on an empty stomach with a beverage containing vitamin C. In one example, the collagen composition is formulated into 500 mg two-piece hard-shell capsules and 2-4 capsules are taken daily. In one example, 1,000 mg of the collagen composition is formulated into a liquid and 1-2 servings are taken daily. In one example, 500 mg is formulation into a drink mix powder and 2-4 servings are taken daily.

The collagen composition may be mixed with water or a citrus juice prior to ingestion. The preparations described above can be taken indefinitely by individuals affected by connective tissue disorders or by healthy individuals as a preventative agent. If desired, an individual with such a disorder can take the preparation until no further improvement is noted in the disorder.

### 3. Methods of Modulating Telomere Length with Collagen Compositions

Provided herein is a method of modulating telomere length by using a collagen composition disclosed herein. The collagen composition may be used to one or more of increase telomerase activity, increase average telomere length, increase proliferative capacity, and reduce the rate of telomere shortening in a cell, which may be of a subject. The increase in telomerase activity, average telomere length or proliferative activity, or reduction in the rate of telomere shortening may be in comparison to a population of control or untreated cells, or to cells from a subject with a condition associated with reduced telomere length. The collagen composition may also be used to increase the lifespan of a cell line (that is, the number of times the cells can divide), such as by increasing the Hayflick limit of the cells.

The cell may be any cell, such as a yeast cell or a mammalian cell. The cell may be a hamster, mouse, rat, monkey, ape, or human cell. The cell may be of the skin, connective tissue, bone marrow, lungs, or gastrointestinal tract. In particular, the cell may be an epithelial cell, epidermal cell, myocardial cell, bone marrow cell, B lymphocyte, T lymphocyte, leukocyte, embryonic stem cell, fibroblast, chondrocyte, or osteoblast. The cell may be under oxidative stress, or may be in contact with reactive oxygen species. The cell may be known in the art for use in a stem cell therapy.

The method may comprise contacting the cell with the collagen composition, where the cell may be in a cell culture or in a subject (e.g., a human subject or human patient in need thereof). The collagen composition may be incorporated into a cell growth medium. In one example, the collagen composition is provided as a powder, which is dissolved in a solution such as PBS or DMSO prior to addition to the cell medium.

Also provided herein is a method of treating or preventing a condition associated with reduced (shortened) telomere length in a subject. The method may comprise administering the collagen composition to a subject, who may be in need thereof. Additionally, provided herein is the use of the collagen composition in the manufacture of a medicament for treating or preventing the condition. Further provided is the collagen composition for use in treating or preventing the condition.

The condition associated with reduced telomere length may be aging, chronic fatigue, depression, senescence, senility, dementia, bone marrow failure/hematopoietic dysfunction, aplastic anemia (which may be acquired), lung and/or liver fibrosis, cancer (including colorectal, esophageal, hematologic cancers, head and neck squamous-cell carcinoma, skin and anorectal cancers, myelodysplastic syndrome, leukemia, and acute myeloid leukemia), inflammatory bowel disease, ulcerative colitis, Barrett's esophagus, cardiovascular disease (including coronary artery disease and myocardial infarction), a degenerative disease associated with aging, or a telomere syndrome, which may be inherited, or one or more known symptoms of the foregoing. The telomere syndrome may be associated with a mutation in telomerase reverse transcriptase gene (TERT), telomerase RNA component (known as TERC or TR), dyskerin (DKC1; located on the sex-linked X chromosome), NOP10, NPH2, TINF2, TCAB1, or RTEL1.

The condition may be dyskeratosis congenita or Hoyeraal-Hreidarsson syndrome; Revesz syndrome; cerebroretinal microangiopathy; Fanconi anemia; cryptogenic cirrhosis; nodular regenerative hyperplasia; Rothmund-Thomson syndrome; Immunodeficiency, centromeric region instability, and facial anomalies type I; or, fascioscapulohumeral muscular dystrophy, or one or more known symptoms of the foregoing. The subject may have bone marrow failure, may be post-chemotherapy, or may be undergoing or have undergone a hematopoietic stem cell transplant. The subject may have been exposed to inflammation, toxins, or radiation exposure.

The collagen composition may also be used to promote at least one of healthy aging, longevity, and wellness in a subject. The collagen composition may be used in a method comprising administering the collagen composition to a subject.

The collagen composition may also be used as an antiaging therapy in a subject. The collagen composition may be used in a method comprising administering the collagen composition to a subject.

The subject may be healthy or may have a condition described herein. The subject may be a mammal, which may be a mouse, rat, dog, cat, pig, horse, cow, monkey, ape, or a human. In particular, the subject may be a human patient.

The present invention has multiple aspects, illustrated by the following non-limiting examples.

### EXAMPLE 1

### Toxicity Assay (MTT) and Telomerase Activity Study (QTRAP) for a Collagen Composition

This example demonstrates that the collagen composition (also referred to as “BCC” in the examples) increases telomerase activity. The experiments determined the toxicity by MTT assay of the collagen composition and evaluation of its effect on telomerase activity by Q-TRAP in cultures of human adult primary fibroblast cells.

### Technique Overview

The MTT toxicity test is a colorimetric assay to measure cells' metabolic activity by serving as a substrate of cellular enzymes that reduce the tetrazolium orange dye, MTT (3-(4,5-Dimethylthiazol-2-yl)-2,5-diphenyltetrazolium bromide) to its insoluble formazan, giving a purple colour. The rate of conversion is directly proportional to cells' mitochondrial metabolic activity via NAD (P) H-dependent cellular oxidoreductase enzymes and serves as a surrogate marker of cell viability.

### Cells and Culture Conditions

Primary cultures of adult human fibroblast working stock (<3 passages) and HeLa cells were established under standard culture conditions. Cells were seeded at 3×10³ cells/cm² in fibroblast medium kit (Innoprot). Fibroblast Medium (FM) is a complete medium designed for optimal growth of normal human fibroblasts in vitro. It is a sterile, liquid medium which contains essential and non-essential amino acids, vitamins, organic and inorganic compounds, hormones, growth factors, trace minerals and a low concentration of fetal bovine serum (2%). The medium is HEPES and bicarbonate buffered and has a pH of 7.4 when equilibrated in an incubator with an atmosphere of 5% CO₂/95% air. The medium is formulated (quantitatively and qualitatively) to provide a defined and optimally balanced nutritional environment that selectively promotes proliferation and growth of normal human fibroblasts in vitro.

### Toxicity Assay (MTT)

Cells previously expanded were seeded in 96 well plates (Nunc) at 0.5×10⁴ cells/plate and 0.35×104 cells/plate for 72 hours and one-week treatment. This concentration is known to show the best window for the MTT assay, with better sensitivity and low variability. The Test Material BCC (collagen composition) was supplied by BioCell Technology LLC as an off-white to beige fine powder. The powder was kept in optimal conditions until its use. A quantity of 29.2 mg of the collagen composition (BCC) was dissolved in 2.9 ml (10 mg/ml) of DMSO 20% at RT. Eight-point curves, ⅓ dilutions, (highest concentration 0.25 mg/ml) were prepared. Final concentration of DMSO was 0.5% (this concentration does not affect the cells) Twenty-four hours following the seeding process, the cells were washed once with PBS and treated with the respective compounds in cell culture media (fibroblast medium kit). Each condition was tested in triplicates with and without 10 μM H₂O₂. For positive and negative controls, 8 mM methyl methane sulfonate (MMS) and DMSO 100% were used respectively.

Following compound addition, the plates were incubated for 72 hours and one week, while the medium with the compound was changed each two days. After the treatment period, cells were washed twice with PBS and media was replaced with MTT reagent at 0.5 mg/ml in DMEM without phenol red. The plates were gently shaken and incubated for 4 hours. After the incubation the medium was removed and replaced by DMSO. The plates were gently shaken to solubilize the formazan crystals. Absorbance was measured using an Envision multiplate reader at a wavelength of 570 nm.

### Results

Toxicity Assay (MTT) in primary fibroblast cells at 72 hours and one week Eight serial dilutions were prepared (250 μg/ml-83.3 μg/ml-27.8 μg/ml-9.26 μg/ml-3.1 μg/ml-1μg/ml -0.34 μg/ml-0.11 μg/ml) to evaluate the compound. Triplicates per concentration were done to evaluate the compound and tested in standard condition and oxidative condition. MTT results showing cell death values above 20% are considered as having a significant toxic effect. The results are shown in Tables 5-8.

TABLE 5
72 hours - Standard condition

| | Cell | Cell | Cell | | |
|---|---|---|---|---|---|
| Concentration | death #1 | death #2 | death #3 | | |
| (μg/ml) | (%) | (%) | (%) | MEAN | SD |
| 250 | −4.0 | 3.4 | −5.5 | −2.0 | 4.8 |
| 83.3 | 9.1 | 0.1 | −1.4 | 2.6 | 5.7 |
| 27.8 | 1.1 | −2.4 | −9.9 | −3.7 | 5.6 |
| 9.26 | 12.6 | 5.8 | 2.4 | 6.9 | 5.2 |
| 3.09 | −1.8 | −16.4 | −13.1 | −10.4 | 7.6 |
| 1.03 | 7.4 | 1.8 | −14.5 | −1.8 | 11.4 |
| 0.34 | −5.6 | −6.1 | −11.5 | −7.7 | 3.3 |
| 0.11 | 12.2 | 5.5 | 3.4 | 7.0 | 4.6 |

TABLE 6
72 hours - Oxidative condition

| | Cell | Cell | Cell | | |
|---|---|---|---|---|---|
| Concentration | death #1 | death #2 | death #3 | | |
| (μg/ml) | (%) | (%) | (%) | MEAN | SD |
| 250 | −7.7 | −10.6 | −10.6 | −9.6 | 1.7 |
| 83.3 | 15.7 | 17.3 | 9.3 | 14.1 | 4.3 |
| 27.8 | −2.6 | −10.3 | −13.2 | −8.7 | 5.5 |
| 9.26 | 6.2 | −1.1 | 1.8 | 2.3 | 3.7 |
| 3.09 | −3.3 | −6.1 | −6.6 | −5.3 | 1.8 |
| 1.03 | −31.7 | −5.2 | −5.9 | −14.3 | 15.1 |
| 0.34 | −5.0 | −7.7 | −10.0 | −7.6 | 2.5 |
| 0.11 | 5.8 | 0.8 | −1.2 | 1.8 | 3.6 |

TABLE 7
1 Week - Standard condition

| | Cell | Cell | Cell | | |
|---|---|---|---|---|---|
| Concentration | death #1 | death #2 | death #3 | | |
| (μg/ml) | (%) | (%) | (%) | MEAN | SD |
| 250 | 4.5 | 7.1 | 2.4 | 4.7 | 2.4 |
| 83.3 | −1.2 | −8.3 | −11.5 | −7.0 | 5.2 |
| 27.8 | −10.9 | −6.8 | 5.3 | −4.1 | 8.4 |
| 9.26 | 0.9 | 0.9 | 3.7 | 1.8 | 1.7 |
| 3.09 | −7.0 | −7.0 | 0.1 | −4.7 | 4.1 |
| 1.03 | 2.7 | 2.7 | 3.2 | 2.9 | 0.3 |
| 0.34 | −1.8 | −3.6 | −4.9 | −3.4 | 1.6 |
| 0.11 | 2.2 | 3.5 | 6.9 | 4.2 | 2.4 |

TABLE 8
1 Week - Oxidative condition

| | Cell | Cell | Cell | | |
|---|---|---|---|---|---|
| Concentration | death #1 | death #2 | death #3 | | |
| (μg/ml) | (%) | (%) | (%) | MEAN | SD |
| 250 | −0.7 | 3.7 | −6.0 | −1.0 | 4.9 |
| 83.3 | −10.4 | −18.5 | −1.0 | −10.0 | 8.8 |
| 27.8 | −32.4 | −43.7 | −42.6 | −39.6 | 6.2 |
| 9.26 | 1.4 | −7.8 | −4.4 | −3.6 | 4.6 |
| 3.09 | −11.7 | −18.8 | −14.9 | −15.1 | 3.5 |
| 1.03 | −1.2 | 8.2 | −2.6 | 1.5 | 5.9 |
| 0.34 | −0.2 | 1.9 | 5.6 | 2.4 | 2.9 |
| 0.11 | 11.1 | 5.8 | 9.5 | 8.8 | 2.7 |

The tables below show the ED₅₀s (Median Effective doses) and % Cell Death at highest concentration for both compounds in primary fibroblast cells.

TABLE 9
Standard condition

| 72 hours | | | One week | |
|---|---|---|---|---|
| | | % Cell Death | | % Cell Death |
| | ED50 | at highest | ED50 | at highest |
| Compound | (μg/mL) | concentration | (μg/mL) | concentration |
| BCC | >250 | −2.0 ± 4.7 | >250 | 4.7 ± 2.4 |

TABLE 10
Oxidative condition

| 72 hours | | | One week | |
|---|---|---|---|---|
| | | % Cell Death | | % Cell Death |
| | ED50 | at highest | ED50 | at highest |
| Compound | (μg/mL) | concentration | (μg/mL) | concentration |
| BCC | >250 | −9.6 ± 1.7 | >250 | −1.0 ± 4.9 |

### Observations and Conclusions

Morphological analysis through optical microscopy visualization (20×) did not show any deleterious effect at any concentration.
No compound precipitation was observed at concentrations tested.
ED50 (Median Effective dose) was found to be >250 μg/ml at both time points (72 hours and one week).
Based on the results, the concentrations chosen for the Q-TRAP and TAT assays are 250, 83.3 and 27.81 μg/ml.

### Telomerase Activity Determination by Q-TRAP

In this assay telomerase activity is determined by quantitative PCR analysis in primary cultures of adult human fibroblast after 6, 24, 48 and 72 hours of treatment and at the concentrations mentioned before.

### Technique Overview

Q-TRAP can measure relative telomerase activity by the Telomeric Repeat Amplification Protocol (TRAP), modified for real-time, quantitative PCR analysis (Q-TRAP). This method has the advantages of exquisite sensitivity, rapidity, and a high-throughput format in respect to the regular TRAP assay. We assess telomerase enzyme activity in whole cell lysates from cell cultures.

The general mechanism of the Q-TRAP technique involves cellular pellets being lysed for protein extraction which is then subsequently quantified and stored under specific conditions to avoid protein degradation. Protein obtained in the process was used inside the following 24 hours, samples were stored at 4° C. Telomerase protein extracts are then incubated with a specific oligonucleotide substrate to allow the enzymatic addition of telomeric DNA repeats by endogenous telomerase.

Following the enzymatic reaction, telomerase extension products are then amplified and quantified by real-time qPCR. In real time PCR, a positive reaction is detected by accumulation of a fluorescent signal. The Ct (cycle threshold) is defined as the number of cycles required for fluorescence to cross the threshold (i.e.: exceeds background levels). The telomerase-positive standard dilution series is plotted against the telomere protein concentration (R2>0.9) as a standard curve of Ct values.

To ensure that the data are both reproducible and quantitative, we routinely perform the assay in triplicate. The mean and standard deviation (SD) from each triplicate is calculated which include both positive (Lymphoid cell line standard Curve) and negative controls (inactivated by heat) are included.

Data are reported as RTA (Relative Telomerase Activity) and the general workflow scheme can be seen below.

PBL cells/cell from different tissues/cell→cellular pellets→protein extraction and quantification→telomerase extension reaction→PCR amplification of telomerase products→real time quantitative PCR (Q-PCR) detection by SYBR GREEN→telomerase activity analysis

### Results

### Quality Control Parameters

Previous to plating and before conducting Q-TRAP protocol samples were assessed for:
Protein concentration: Protein quantification is performed in each sample using Biorad protein assay. A minimum of 0.3 mg/ml protein concentration is required to proceed with the analysis of the samples to guarantee consistent results.
Regression Curve: Internal controls are included and a regression analysis is performed for each run/plate. Those plates in which regression curves have an R2 below 0.9 are repeated.
Replicates: Samples with less than 2 valid replicates are discarded.
Cycle amplification: Signals obtained after cycle #35 are considered unspecific amplification.

The abbreviations used in herein are as follows: Control untreated group (Ctrl);

Treatment with BCC at 250 μg/ml (BCC [250]); Treatment with BCC at 83.3 μg/ml (BCC [83.3]); and, Treatment with BCC at 27.8 μg/ml (BCC [27.8]).

The protein concentrations for samples analyzed by Q-TRAP were measured in triplicate for quality control.

### Telomerase Activity Results

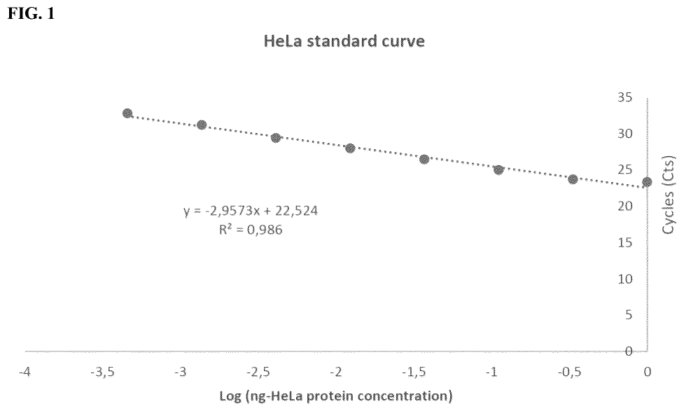

Only those samples with sufficient amount of protein were analyzed (>0.3 mg/ml). Standard curve results: generated by graphing Threshold cycles (Ct values) of HeLa cell line standards against log of 1000, 333, 111, 37.03, 12.34, 4.11, 1.37 and 0.45 ng of protein (whole cell extract). The results are shown in FIG. 1.

Measurements were performed in triplicates to calculate coefficients of variation and mean amplification signals. The table below summarize the average of triplicate data, mean and SD after normalization against Hela control reported as Relative Telomerase Activity (RTA) for each sample.

TABLE 11
RTAs value for each sample analyzed
Telomerase Activity (RTA %)

| | 0 hours | | 6 hours | | 24 hours | | 48 hours | | 72 hours | |
|---|---|---|---|---|---|---|---|---|---|---|
| | RTA | | RTA | | RTA | | RTA | | RTA | |
| Treatment | % | SD | % | SD | % | SD | % | SD | % | SD |
| Control | 3.2 | 1.4 | 40.8 | 1.1 | 16.6 | 1.6 | 26.6 | 0.3 | 6.9 | 0.8 |
| BCC [250] | | | 40.2 | 2.6 | 19.0 | 1.8 | 27.1 | 5.9 | 9.1 | 1.1 |
| BCC [83.3] | | | 46.7 | 1.1 | 23.1 | 2.3 | 32.3 | 7.2 | 7.7 | 0.7 |
| BCC [27.8] | | | 35.6 | 4.7 | 20.6 | 5.2 | 29.0 | 6.1 | 7.5 | 0.3 |

### Data Analysis

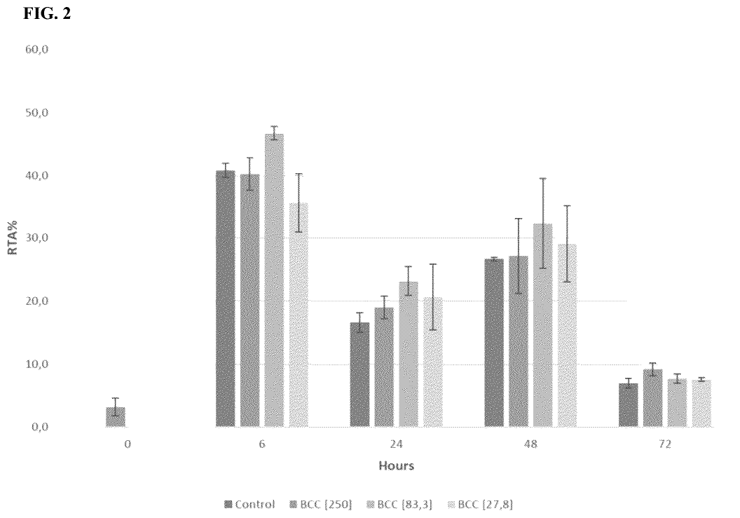

Q-TRAP analysis for human adult primary fibroblasts at different times were compared. Data were grouped by condition and time point of treatment. Statistical analysis for grouped data was applied using t test comparing with control condition. The results are shown in FIG. 2.

The data are further analyzed in the following table. T-Student analysis indicates if there are significant differences among the Q-TRAP results (RTA) compared to control condition. Significant differences are indicated in the “Significance” column. From lowest to highest significance: No: non-significant; Yes (*): p<0.05; Yes (**): p<0.01; Yes (***): p<0.001; Yes (****): n<0.0001.

TABLE 12
T-Student Analysis - Relative Telomerase Activity

| | RTA (p Value) | Significance |
|---|---|---|
| 6 hours | | |
| Ctrl vs. BCC [250] | 8.04E−01 | NO |
| Ctrl vs. BCC [83.3] | 3.33E−02 | YES (*) |
| Ctrl vs. BCC [27.8] | 2.37E−01 | NO |
| 24 hours | | |
| Ctrl vs. BCC [250] | 1.59E−01 | NO |
| Ctrl vs. BCC [83.3] | 3.03E−02 | YES (*) |
| Ctrl vs. BCC [27.8] | 2.70E−01 | NO |
| 48 hours | | |
| Ctrl vs. BCC [250] | 4.40E−01 | NO |
| Ctrl 1 vs. BCC [83.3] | 7.48E−01 | NO |
| Ctrl vs. BCC [27.8] | 6.34E−01 | NO |
| 72 hours | | |
| Ctrl vs. BCC [250] | 4.42E−02 | YES (*) |
| Ctrl vs. BCC [83.3] | 2.98E−01 | NO |
| Ctrl vs. BCC [27.8] | 2.88E−01 | NO |

### Observations and Conclusions

### Quality Control Results

Protein extraction yields from all samples rendered suitable amount of protein (>0.3 mg/ml) to perform the Q-TRAP assay.
The regression curve's coefficient of determination (HeLa Cells) was higher than 0.9 (R2=0.99).

### Telomerase Activity Results

At 6 hours an increase in telomerase activity was observed in the cell group treated with BCC[83.3] compared to the control untreated group. This difference was found to be statistically significant (Table 12).
At 24 hours, an increase in telomerase activity was detected in all treated groups compared to control. The difference between control and the groups treated with BCC[83.3] was found to be statistically significant (Table 12).
At 48 hours an increase in telomerase activity was observed in the cell groups treated with BCC[83.3] and BCC[27.8] compared to control. Although these differences were not identified as significant by the statistical analysis a clear trend in telomerase activation can be observed for group BCC[83.3].

### EXAMPLE 2

### In Vitro Proliferative and Telomere Length Analysis (TAT) for a Collagen Composition

This example demonstrates that the collagen composition increase telomere length. The experiments determine the cellular proliferation rate and telomere length in cultures of human adult primary fibroblast cells treated with the collagen composition.

### Proliferative Analysis in Cell Culture

### Overview

Primary cultures of adult human fibroblast cells were established. Cells are seeded at 5×10³ cells/cm²), in fibroblast medium kit (Innoprot). Fibroblast Medium (FM) is a complete medium designed for optimal growth of normal human fibroblasts in vitro. It is a sterile, liquid medium which contains essential and non-essential amino acids, vitamins, organic and inorganic compounds, hormones, growth factors, trace minerals and a low concentration of foetal bovine serum (2%). The medium is HEPES and bicarbonate buffered and has a pH of 7.4 when equilibrated in an incubator with an atmosphere of 5% CO₂/95% air. The medium is formulated (quantitatively and qualitatively) to provide a defined and optimally balanced nutritional environment that selectively promotes proliferation and growth of normal human fibroblasts in vitro.

Media is renewed every 2-3 days and cells passaged at sub-confluence (70-80%) every 7 days. Compounds or vehicle control are added to the cells in culture. Cell growth is monitored for each condition by counting cell numbers at each passage using a COUNTESSTM cell counter (Invitrogen). Population doubling (PD) was calculated with the formula PD=3.322(Log (Cf)−Log(Ci))+x(Cf: Final concentration; Ci: initial concentration; X: PD last passage). One PD is equivalent to one round of cell replication.

### Materials and Methods

The Test Material BCC (collagen composition) was supplied by BioCell Technology LLC as an off-white to beige fine powder.

### Treatments

Independent conditions were evaluated during the proliferative analysis:
Standard cell culture condition (Ctrl)
Standard cell culture condition+BCC [250]
Standard cell culture condition+BCC [83.3]
Standard cell culture condition+BCC [27.8]
Oxidative cell culture condition=Standard cell culture condition+10 μM H₂O₂ (H₂O₂ )
Oxidative cell culture condition+BCC [250]+H₂O₂
Oxidative cell culture condition+BCC [83.3]+H₂O₂
Oxidative cell culture condition+BCC [27.8]+H₂O₂

Cells were expanded during eight weeks under standard and oxidative (10 μM H₂O₂ ) cell culture condition as described previously. Treatment dilutions were prepared fresh in each passage with fresh media. The collagen composition (BCC) was diluted in DMEM medium to add to the cells. A dilution of 1/40 was prepared.

### Results

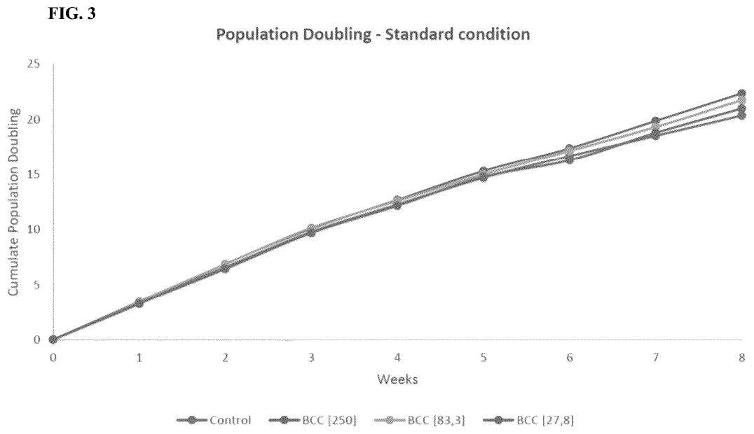

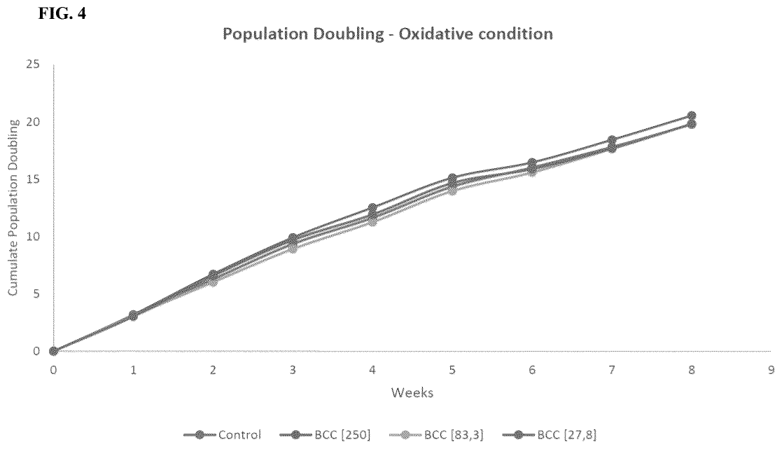

The results are shown in Tables 13 and 14, and in FIGS. 3 and 4.

TABLE 13
Cumulative Population Doubling per passage
(average of three measurements) in standard condition
Cumulative population doubling

| Week | 0 | 1 | 2 | 3 | 4 | 5 | 6 | 7 | 8 |
|---|---|---|---|---|---|---|---|---|---|
| Control | 0 | 3.3 | 6.6 | 9.8 | 12.2 | 14.7 | 16.7 | 18.5 | 20.3 |
| BCC [250] | 0 | 3.2 | 6.9 | 10.1 | 12.7 | 15.3 | 17.4 | 19.9 | 22.3 |
| BCC [83.3] | 0 | 3.5 | 6.9 | 10.2 | 12.6 | 15.0 | 17.1 | 19.3 | 21.7 |
| BCC [27.8] | 0 | 3.3 | 6.4 | 9.7 | 12.1 | 14.8 | 16.3 | 18.8 | 21.0 |

TABLE 14
Cumulative Population Doubling per passage
(average of three measurements) in oxidative conditions
Cumulative population doubling

| Week | 0 | 1 | 2 | 3 | 4 | 5 | 6 | 7 | 8 |
|---|---|---|---|---|---|---|---|---|---|
| H₂O₂ | 0 | 3.14 | 6.26 | 9.33 | 11.61 | 14.33 | 15.99 | 17.78 | 19.77 |
| BCC | 0 | 3.19 | 6.72 | 9.90 | 12.52 | 15.11 | 16.45 | 18.42 | 20.53 |
| [250] + | | | | | | | | | |
| 10 μM | | | | | | | | | |
| H₂O₂ | | | | | | | | | |
| BCC | 0 | 3.07 | 6.01 | 8.90 | 11.23 | 13.94 | 15.56 | 17.63 | 19.81 |
| [83.3] + | | | | | | | | | |
| 10 μM | | | | | | | | | |
| H₂O₂ | | | | | | | | | |
| BCC | 0 | 3.02 | 6.55 | 9.67 | 11.91 | 14.64 | 15.86 | 17.65 | 19.79 |
| [27.8] + | | | | | | | | | |
| 10 μM | | | | | | | | | |
| H₂O₂ | | | | | | | | | |

### Observations and Conclusions

After 8 weeks of treatment at the predetermined concentrations

Under standard culture conditions:
Cells treated with BCC presented an increase in proliferative capacity after the second week of expansion.

Under oxidative culture conditions:
Cells treated with BCC[250] presented an increase in proliferative capacity after the second week of expansion.

### telomere Length Measurements By TAT®

### Technique Overview

For the measurement of the median telomere length of any cell line, a high throughput (HT) Q-FISH technique was used. This method is based on a quantitative fluorescence in-situ hybridization method modified for cells in interphase. In brief, telomeres are hybridized with a fluorescent Peptide Nucleic Acid probe (PNA) that recognizes three telomere repeats (sequence: Alexa488-OO-CCCTAACCCTAACCCTAA (SEQ ID NO: 1), Panagene). The images of the nuclei and telomeres are captured by a high-content screen system (see below). The intensity of the fluorescent signal from the telomeric PNA probes that hybridize to a given telomere is proportional to the length of that telomere. The intensities of fluorescence are translated to base pairs through a standard regression curve which is generated using control cell lines with known telomere length.

Sample Preparation and HT Q-FISH: On processing day, the samples and control cell lines frozen in liquid nitrogen are thawed at 37° C. and cell counts and cellular viability are determined. Aliquots with viability lower than 60% are considered below our QC standards and are not further analyzed. Cells are seeded in clear bottom black-walled 384-well plates at the density of 15,000 cells per well with 5 replicates of each sample and 8 replicates of each control cell line. Two identical independent plates are prepared for each set of samples. Cells are fixed with methanol/acetic acid (3/1, vol/vol). Once these cells have fixed onto the plate, they are treated with pepsin to digest the cytoplasm and the nuclei are processed for in situ hybridization with the PNA probe. After several washing steps following standard DAPI incubation for DNA staining, the wells are filled up with mounting medium and the plate is stored overnight at 4° C.

HT Microscopy: Quantitative image acquisition and analysis is performed on a High Content Screening Opera Phenix System (Perkin Elmer), using the Columbus software, Version 2.9 (Perkin Elmer). Images are captured, using a 40×0.95 NA water immersion objective. UV and 488 nm excitation wavelengths are used to detect the DAPI and A488 signals respectively. With constant exposure settings, 15 independent images are captured at different positions for each well. Next, the nuclei images are used to define the region of interest for each cell, measuring telomere fluorescence intensity of the A488 image in all of them. The results of intensity for each foci are exported to the Columbus 2.4 software (Perkin Elmer). Telomere length distribution and median telomere length are calculated with Life Length's proprietary program. Statistical analysis of the data was performed using T-Student test.

### Technology Validation

The TAT technology has been validated for the following parameters:

Accuracy: The establishment of a correspondence between TAT fluorescence intensity values and telomere length measurements is achieved by performing TRF (Terminal Restriction Fragmentation) in six human lymphocyte cell lines (Calibration / Method Comparison). The same set of samples is analyzed both by TAT and by the TRF reference method (Definition of TAT Systemic Error). VALIDATION DATA shows a correlation of 0.99.

Precision: Serial analysis of the median telomere length values is performed on a human lymphocyte sample in different runs, days and plate positions in order to define TAT Random Error parameters (Standard Deviation, Variance). VALIDATION DATA indicates that TAT has a standard deviation of 454 base pairs.

Limit of Detection and Specificity: Definition of image analysis algorithms and protocol settings exist that define the lowest significant spot intensities and avoid interference by nonspecific fluorescence signals. VALIDATION DATA defines the limit of detection at 800 base pairs and demonstrates very high specificity.

Median Reportable Range: Analysis of median telomere length of 6 cell lines is performed that covers our reportable range and defines its lower and upper limits. VALIDATION DATA fix lower level at 4,700 base pairs and upper level at 14,400 base pairs.

Reference Range: Analysis of median telomere length have been conducted in hundreds of human samples in order to define the TAT Reference Range and its percentiles (5th, 10th, 25th, 50th, 75th and 95th) for different ages. VALIDATION DATA established population curves-normal population data base from 18 to 85 years, to extrapolate patients' data and generate reports.

### Results

### Quality Control (QC) Parameters

During the TAT protocol, the samples were assessed for:
Cell count: An automated cell counter is used to determine the total number of cells in the vials.
Cell viability by Trypan-Blue exclusion method.
Regression Curve: Internal controls are included and a regression analysis is performed for each run/plate. The plates are repeated if their regression curves have an R2 below 0.92.
Replicates: After seeding and once TAT is completed:
Samples with CV above 10% are discarded.
Samples with less than 3 valid replicates at the end of the analysis are discarded.
Spot number analyzed per sample should be higher than 10,000.

The concentration and viability of samples after thawing of triplicates in standard and oxidative conditions were measured for quality control.

Telomere LENGTH RESULTS from TAT analysis

The following tables show the median telomere length and 20th percentile median telomere length (both in base pairs-bp) for each sample as well as the percentage of short telomeres. The latter is defined as the percentage of the telomeres with a length below 3 Kbp (<3 Kbp). All measurements were performed in quintuplicate.

TABLE 15
Telomere length results from TAT analysis (standard conditions)

| | | | 20th | | |
|---|---|---|---|---|---|
| | | Median | Percentile | Telomeres < | |
| | Treatment and Time | Length | Length | 3 Kbp | CV |
| LL Code | Point | (base pairs) | (base pairs) | (%) | (%) |
| Ctrl-P0 | Control-Week 0 | 10148 | 6468 | 4.0 | 3.9 |
| Control-P2 | Control-Week 2 | 10321 | 6186 | 6.2 | 3.5 |
| BCC [250]-P2 | BCC [250]-Week 2 | 10451 | 6306 | 5.7 | 0.9 |
| BCC [83.3]-P2 | BCC [83.3]-Week 2 | 10259 | 6108 | 6.5 | 3.0 |
| BCC [27.8]-P2 | BCC [27.8]-Week 2 | 10281 | 6125 | 6.3 | 2.1 |
| Control-P4 | Control-Week 4 | 9420 | 5757 | 6.2 | 0.5 |
| BCC [250]-P4 | BCC [250] - Week 4 | 9519 | 5823 | 6.1 | 2.4 |
| BCC [83.3]-P4 | BCC [83.3]-Week 4 | 9441 | 5867 | 5.9 | 1.6 |
| BCC [27.8]-P4 | BCC [27.8]-Week 4 | 9734 | 6058 | 5.4 | 0.6 |
| Control-P6 | Control-Week 6 | 8780 | 4590 | 11.8 | 0.9 |
| BCC [250]-P6 | BCC [250]-Week 6 | 8974 | 4832 | 10.5 | 1.0 |
| BCC [83.3]-P6 | BCC [83.3]-Week 6 | 9042 | 4916 | 10.3 | 3.8 |
| BCC [27.8]-P6 | BCC [27.8]-Week 6 | 9147 | 4932 | 10.2 | 2.5 |
| Control-P8 | Control-Week 8 | 8022 | 4474 | 11.4 | 4.7 |
| BCC [250]-P8 | BCC [250]- Week 8 | 7987 | 4524 | 10.5 | 1.8 |
| BCC [83.3]-P8 | BCC [83.3]-Week 8 | 8087 | 4498 | 10.9 | 4.4 |
| BCC [27.8]-P8 | BCC [27.8]-Week 8 | 8484 | 4921 | 9.1 | 7.5 |

TABLE 16
Telomere length results from TAT analysis (oxidative conditions)

| | | 20th | | | |
|---|---|---|---|---|---|
| | Median | Percentile | Telomeres < | | |
| LL Code Treatment and | Length | Length | 3 Kbp | CV | |
| Time Point | (base pairs) | (base pairs) | (%) | (%) | |
| Control-P2 | 10 μM H₂O₂-Week 2 | 10115 | 5975 | 6.7 | 0.5 |
| BCC[250] + 10 μM H₂O₂ | BCC[250] + 10 μM | 10134 | 5929 | 7.0 | 0.8 |
| | H₂O₂-Week 2 | | | | |
| BCC[83.3] + 10 μM H₂O₂ | BCC[83.3] + 10μ | 10443 | 6252 | 5.9 | 0.5 |
| | H₂O₂-Week 2 | | | | |
| BCC[27.8] + 10 μM H₂O₂ | BCC[27.8] + 10 μM | 10448 | 6281 | 6.1 | 2.9 |
| | H₂O₂-Week 2 | | | | |
| Control-P4 | 10 μM H₂O₂-Week 4 | 9307 | 5616 | 6.9 | 2.3 |
| BCC[250] + 10 μM H₂O₂ | BCC[250] + 10 μM | 9377 | 5630 | 6.8 | 1.8 |
| | H₂O₂-Week 4 | | | | |
| BCC[83.3] + 10 μM H₂O₂ | BCC[83.3] + 10 μM | 9181 | 5502 | 7.0 | 0.6 |
| | H₂O₂-Week 4 | | | | |
| BCC[27.8] + 10 μM H₂O₂ | BCC[27.8] + 10 μM | 9553 | 5808 | 6.0 | 1.3 |
| | H₂O₂-Week 4 | | | | |
| Control-P6 | 10 μM H₂O₂-Week 6 | 8972 | 4782 | 10.9 | 1.2 |
| BCC[250] + 10 μM H₂O₂ | BCC[250] + 10 μM | 8960 | 4718 | 11.4 | 1.7 |
| | H₂O₂-Week 6 | | | | |
| BCC[83.3] + 10 μM H₂O₂ | BCC[83.3] + 10 μM | 8945 | 4708 | 11.4 | 1.4 |
| | H₂O₂-Week 6 | | | | |
| BCC[27.8] + 10 μM H₂O₂ | BCC[27.8] + 10 μM | 9055 | 4757 | 11.2 | 1.2 |
| | H₂O₂-Week 6 | | | | |
| Control-P8 | 10 μM H₂O₂-Week 8 | 8490 | 4869 | 9.0 | 2.6 |
| BCC[250] + 10 μM H₂O₂ | BCC[250] + 10 μM | 8620 | 4995 | 8.5 | 2.8 |
| | H₂O₂-Week 8 | | | | |
| BCC[83.3] + 10 μM H₂O₂ | BCC[83.3] + 10 μM | 8413 | 4767 | 9.7 | 5.9 |
| | H₂O₂-Week 8 | | | | |
| BCC[27.8] + 10 μM H₂O₂ | BCC[27.8] + 10 μM | 8342 | 4754 | 10.0 | 3.7 |
| | H₂O₂-Week 8 | | | | |

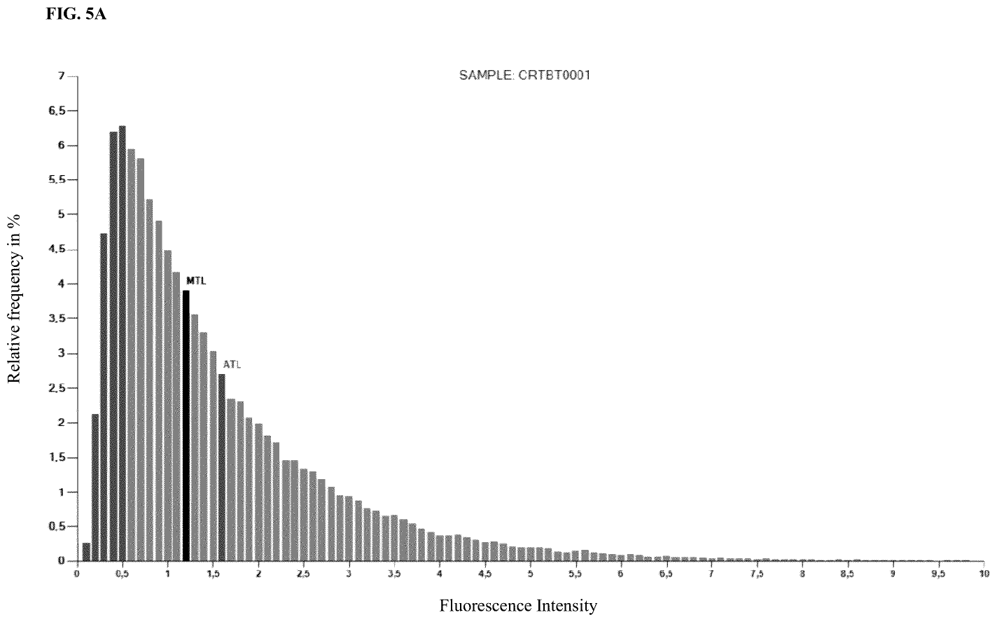

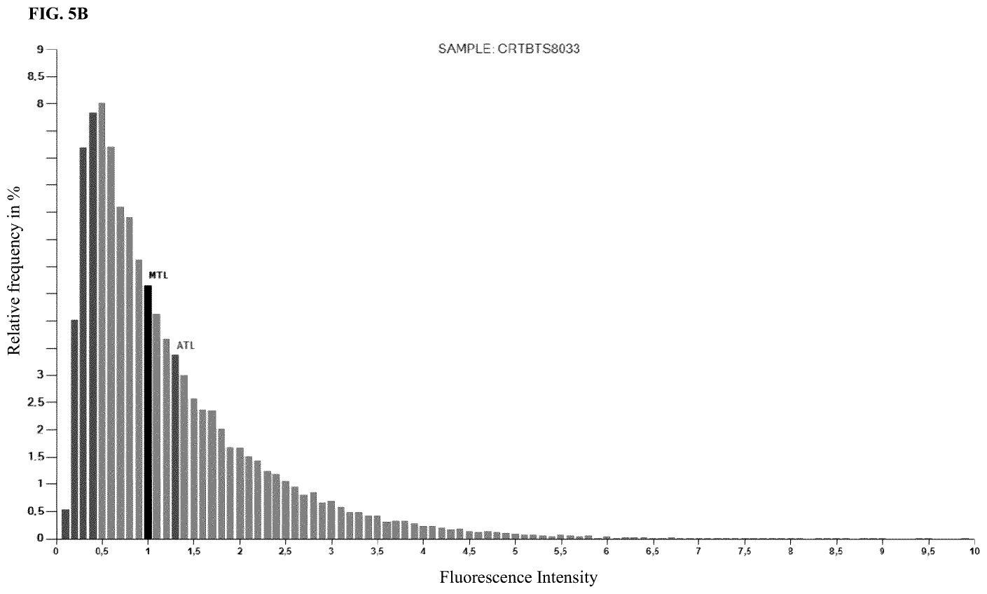

Representative histograms are shown in FIG. 5A and B.

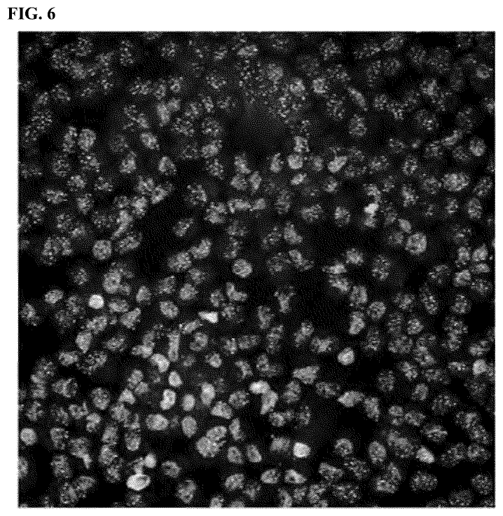

FIG. 6 shows a representative TAT image.

### Data Analysis

For TAT analysis, data were grouped by standard and oxidative with BCC (250, 83.3 and 27.8) and time point of treatment (0, 2, 4, 6 and 8 weeks) for standard and oxidative conditions.

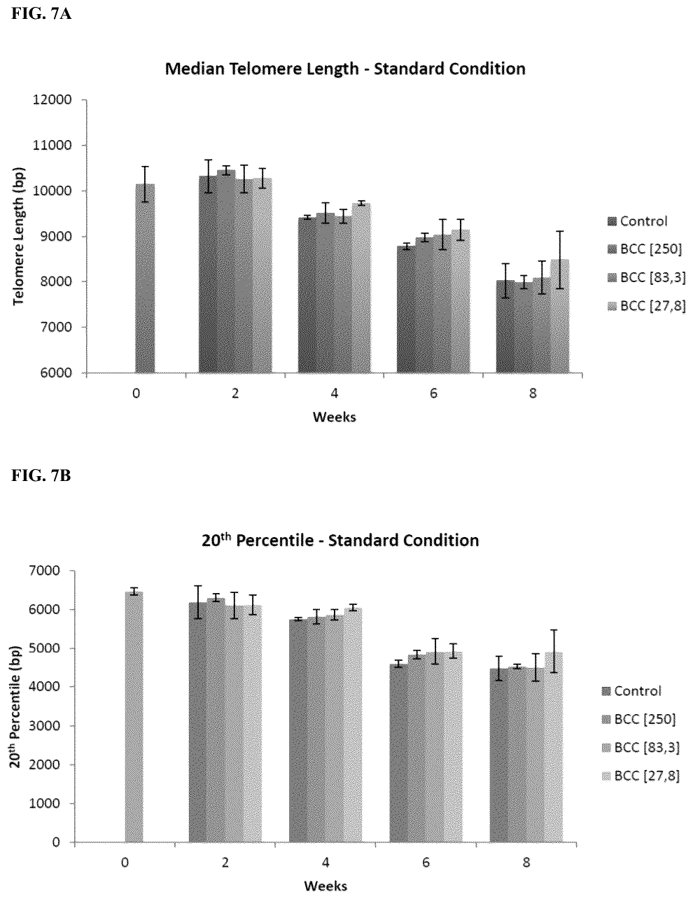

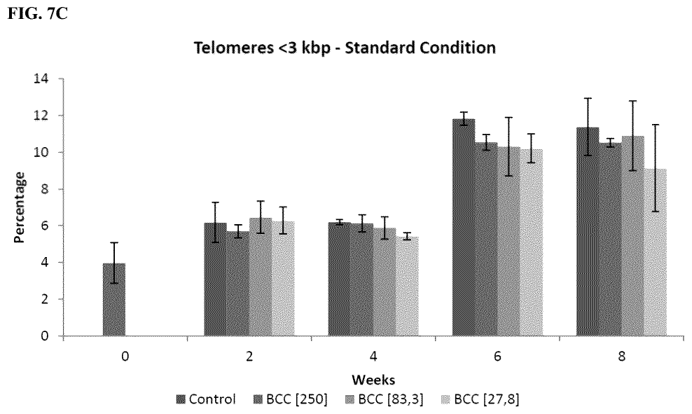

The results for standard cell culture conditions are shown in FIG. 7A-C. The table below shows T-Student analysis indicating if there are significant differences among the treatments in standard conditions. Significant differences are indicated in the “Significance” column. From lowest to highest significance: No: non-significant; Yes (*): p<0.05; Yes (**): p<0.01; Yes (***): p<0.001; Yes (****): p<0.0001.

TABLE 17
T-Student Analysis - Telomere Length Variables - Standard cell culture conditions

| | | | 20th | | | |
|---|---|---|---|---|---|---|
| | Median | | Percentile | | Telomeres < | |
| | Length | | Length | | 3 Kbp | |
| | (p Value) | Significance | (p Value) | Significance | (p value) | Significance |
| Week 2 | | | | | | |
| Control vs. | 1.97E−01 | NO | 2.87E−01 | NO | 1.13E−01 | NO |
| BCC [250] | | | | | | |
| Control vs. | 6.01E−01 | NO | 5.51E−01 | NO | 4.22E−01 | NO |
| BCC [83.3] | | | | | | |
| Control vs. | 7.21E−01 | NO | 6.26E−01 | NO | 7.85E−01 | NO |
| BCC [27.8] | | | | | | |
| Week 4 | | | | | | |
| Control vs. | 3.87E−01 | NO | 5.80E−01 | NO | 8.71E−01 | NO |
| BCC [250] | | | | | | |
| Control vs. | 8.03E−01 | NO | 2.63E−01 | NO | 3.02E−01 | NO |
| BCC [83.3] | | | | | | |
| Control vs. | 3.83E−03 | YES (**) | 8.68E−03 | YES (*) | 1.07E−02 | YES (*) |
| BCC [27.8] | | | | | | |
| Week 6 | | | | | | |
| Control vs. | 3.48E−02 | YES (*) | 1.22E−02 | YES (**) | 4.62E−03 | YES (***) |
| BCC [250] | | | | | | |
| Control vs. | 2.97E−02 | YES (*) | 7.49E−03 | YES (***) | 6.64E−03 | YES (***) |
| BCC [83.3] | | | | | | |
| Control vs. | 3.02E−02 | YES (*) | 1.31E−03 | YES (***) | 7.94E−04 | YES (***) |
| BCC [27.8] | | | | | | |
| Week 8 | | | | | | |
| Control vs. | 8.17E−01 | NO | 8.41E−01 | NO | 4.78E−01 | NO |
| BCC [250] | | | | | | |
| Control vs. | 7.49E−01 | NO | 8.79E−01 | NO | 5.20E−01 | NO |
| BCC [83.3] | | | | | | |
| Control vs. | 7.29E−01 | NO | 2.51E−02 | YES (*) | 1.01E−02 | YES (**) |
| BCC [27.8] | | | | | | |

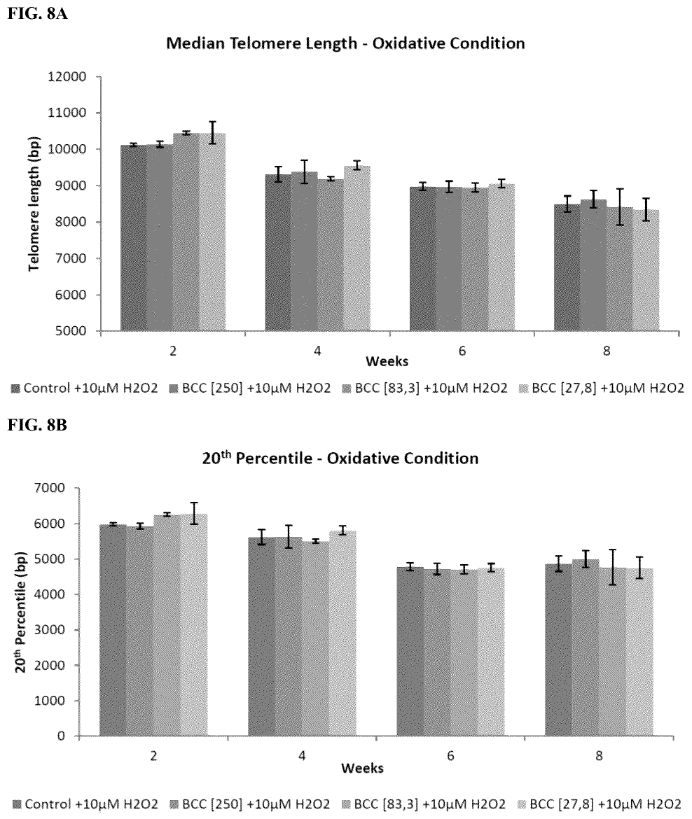

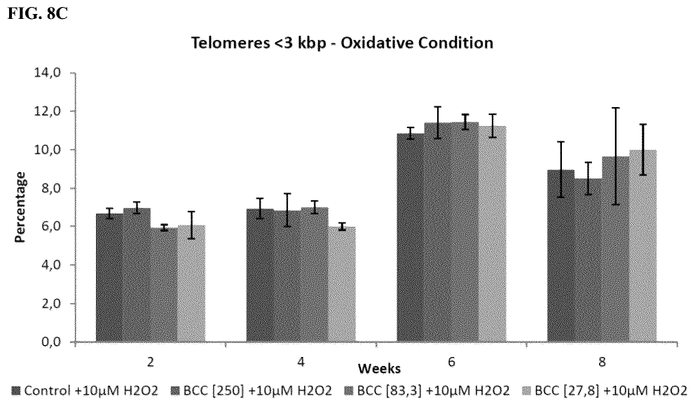

The results for oxidative cell culture conditions are shown in FIG. 8A-C. The table below shows T-Student analysis indicating if there are significant differences among the treatments in oxidative conditions. Significant differences are indicated in the “Significance” column. From lowest to highest significance: No: non-significant; Yes (*): p<0.05; Yes (**): p<0.01; Yes (***): p<0.001; Yes (****): p<0.0001.

TABLE 18
T-Student Analysis - Telomere Length Variables - Oxidative cell culture conditions

| | | | 20th | | | |
|---|---|---|---|---|---|---|
| | Median | | Percentile | | Telomeres < | |
| | Length | | Length | | 3 Kbp | |
| | (p Value) | Significance | (p Value) | Significance | (p value) | Significance |
| Week 2 | | | | | | |
| Control vs. | 1.97E−02 | YES (*) | 6.93E−02 | YES (**) | 1.93E−01 | YES (**) |
| BCC [250] | | | | | | |
| Control vs. | 6.21E−06 | YES (****) | 1.38E−04 | YES (****) | 1.80E−03 | YES (**) |
| BCC [83.3] | | | | | | |
| Control vs. | 1.38E−03 | YES (**) | 1.34E−02 | NO | 1.24E−01 | NO |
| BCC [27.8] | | | | | | |
| Week 4 | | | | | | |
| Control vs. | 2.27E−02 | YES (*) | 3.58E−02 | YES (*) | 4.41E−02 | NO |
| BCC [250] | | | | | | |
| Control vs. | 2.73E−01 | NO | 3.53E−01 | NO | 9.49E−01 | NO |
| BCC [83.3] | | | | | | |
| Control vs. | 3.22E−02 | YES (*) | 4.10E−02 | YES (*) | 5.91E−03 | YES (**) |
| BCC [27.8] | | | | | | |
| Week 6 | | | | | | |
| Control vs. | 7.98E−01 | NO | 5.35E−01 | NO | 2.75E−01 | NO |
| BCC [250] | | | | | | |
| Control vs. | 7.04E−01 | NO | 4.50E−01 | NO | 2.55E−01 | NO |
| BCC [83.3] | | | | | | |
| Control vs. | 5.09E−01 | NO | 6.55E−01 | NO | 2.95E−01 | NO |
| BCC [27.8] | | | | | | |
| Week 8 | | | | | | |
| Control vs. | 3.35E−01 | NO | 3.52E−01 | NO | 5.06E−01 | NO |
| BCC [250] | | | | | | |
| Control vs. | 6.25E−02 | NO | 9.89E−02 | NO | 7.57E−02 | NO |
| BCC [83.3] | | | | | | |
| Control vs. | 2.58E−01 | NO | 3.94E−01 | NO | 1.65E−01 | NO |
| BCC [27.8] | | | | | | |

Because cell replication is one of the principal causes of telomere shortening, the telomere length measurements performed were normalized by the population doubling levels (cell replication) in each condition and time point.

The table below shows telomere shortening rate (Median of telomere length (initial-final) /Population Doubling) at the defined time points and treatments under standard condition.

TABLE 19

| Treatment | Week 2 | Week 4 | Week 6 | Week 8 |
|---|---|---|---|---|
| Control | 48.5 | 82.7 | 99.3 | 118.6 |
| BCC [250] | −10.6 | 71.9 | 83.9 | 109.4 |
| BCC [83.3] | 2.1 | 78.8 | 91.6 | 117.2 |
| BCC [27.8] | 0.4 | 57.7 | 85.8 | 77.2 |

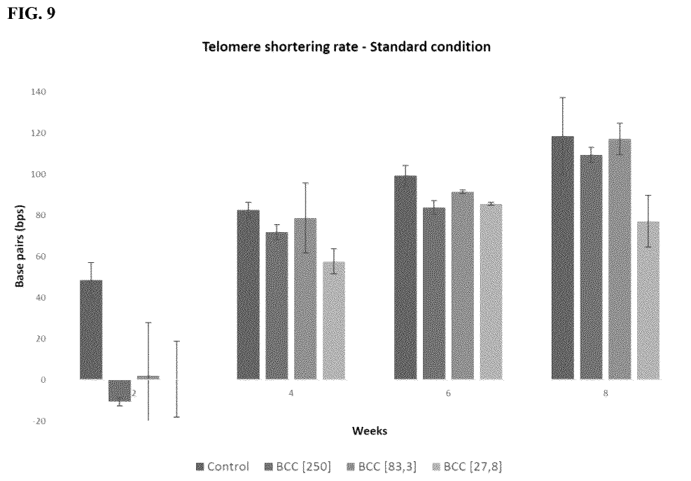

FIG. 9 shows bar charts of shortening rate for different time points and treatments under standard conditions.

The table below shows T-Student analysis indicating if there are significant differences among the Telomere Shortening Rates observed in each treatment under standard condition. Significant differences are indicated in the “Significance” column. From lowest to highest significance: No: non-significant; Yes (*): p<0.05; Yes (**): p<0.01; Yes (***): p<0.001; Yes (****): p<0.0001.

TABLE 20
T-Student Analysis - Telomere Shortening
Rate - Standard condition

| | p Value | Significance |
|---|---|---|
| Week 2 | | |
| Control vs. BCC [250] | 0.01 | YES (*) |
| Control vs. BCC [83.3] | 0.14 | NO |
| Control vs. BCC [27.8] | 0.08 | NO |
| Week 4 | | |
| Control vs. BCC [250] | 0.34 | NO |
| Control vs. BCC [83.3] | 0.59 | NO |
| Control vs. BCC [27.8] | 0.004 | YES (**) |
| Week 6 | | |
| Control vs. BCC [250] | 0.012 | YES (*) |
| Control vs. BCC [83.3] | 0.130 | NO |
| Control vs. BCC [27.8] | 0.036 | YES (*) |
| Week 8 | | |
| Control vs. BCC [250] | 0.45 | NO |
| Control vs. BCC [83.3] | 0.93 | NO |
| Control vs. BCC [27.8] | 0.08 | NO |

The table below shows telomere shortening rate (Median of telomere length (initial-final)/Population Doubling) at the defined time points and treatments under oxidative condition.

TABLE 21

| Treatment | Week 2 | Week 4 | Week 6 | Week 8 |
|---|---|---|---|---|
| Control + 10 μM H2O2 | 50.8 | 109.7 | 109.7 | 98.5 |
| BCC [250] + 10 μM H2O2 | 43.8 | 85.9 | 73.8 | 82.6 |
| BCC [83.3] + 10 μM H2O2− | 1.9 | 109.7 | 91.3 | 82.6 |
| BCC [27.8] + 10 μM H2O2 | 22.6 | 85.9 | 78.8 | 104.3 |

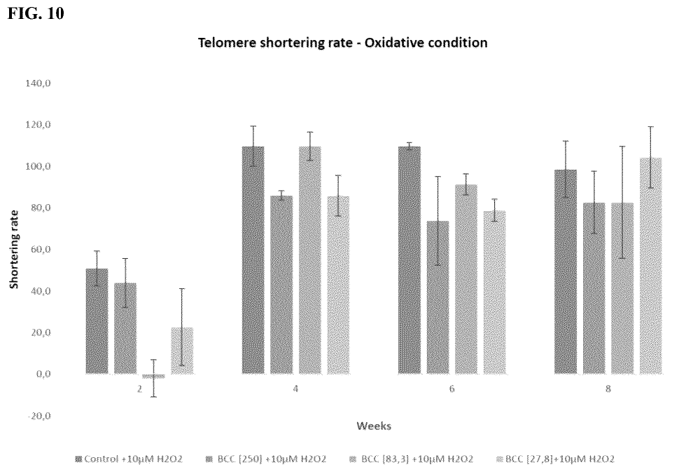

FIG. 10 shows bar charts of shortening rate for different time points and treatments under oxidative conditions.

The table below shows T-Student analysis indicating if there are significant differences among the Telomere Shortening Rates observed in each treatment. Significant differences are indicated in the “Significance” column. From lowest to highest significance: No: non-significant; Yes (*): p<0.05; Yes (**): p<0.01; Yes (***): p<0.001; Yes (****): p<0.0001.

TABLE 22
T-Student Analysis -Telomere Shortening
Rate - Oxidative conditions

| | p Value | Significance |
|---|---|---|
| Week 2 | | |
| Control vs. BCC [250] | 4.88E−01 | NO |
| Control vs. BCC [83.3] | 1.68E−03 | YES (**) |
| Control vs. BCC [27.8] | 9.26E−02 | NO |
| Week 4 | | |
| Control vs. BCC [250] | 7.68E−02 | NO |
| Control vs. BCC [83.3] | 9.99E−01 | NO |
| Control vs. BCC [27.8] | 2.72E−02 | YES (*) |
| Week 6 | | |
| Control vs. BCC [250] | 3.50E−01 | NO |
| Control vs. BCC [83.3] | 8.50E−01 | NO |
| Control vs. BCC [27.8] | 6.79E−02 | NO |
| Week 8 | | |
| Control vs. BCC [250] | 4.48E−01 | NO |
| Control vs. BCC [83.3] | 7.77E−01 | NO |
| Control vs. BCC [27.8] | 6.44E−01 | NO |

### Observations and Conclusions

### Standard Cell Culture Conditions

Almost all treated groups are found to have a higher median telomere length and 20th percentile length as well as a lower percentage of telomeres <3kpb compared to untreated control group. This was visible in weeks 4, 6, and 8.
The statistical analysis detected that all differences, between control and treated groups, were significant at 6 weeks of treatment. The group treated with BCC [27.8] demonstrated significant differences compared to control in weeks 4 and 8 apart from week 6.
After normalizing the data by the population doubling it was observed that all concentrations lowered the telomere shortening rate throughout the entire expansion, suggesting a protective effect on telomere length. Statistical differences were identified for concentrations BCC at 2 and 6 weeks and BCC [27.8] at 4 and 6 weeks of treatment.

### Oxidative Cell Culture Conditions

Higher median telomere length and 20th percentile length values as well as lower percentages of telomeres <3kpb were observed for groups treated BCC [250], BCC [83.3] and BCC [27.8] at 2 weeks and groups treated with BCC and BCC [27.8] at 4 weeks. These differences were found to be significant by the statistical analysis.

After normalizing the data by the population doubling it was observed that all concentrations (with the exception of BCC [83.3] at week 4 and BCC [27.8] at week 8) lowered the telomere shortening rate throughout the entire expansion, suggesting a protective effect on telomere length.

1. A method of at least one of increasing telomerase activity, increasing average telomere length, reducing the rate of telomere shortening, and increasing proliferative capacity of a cell, comprising contacting the cell with a composition comprising hydrolyzed collagen having an average molecular weight of between about 50 and about 10,000 Da and at least about 20% glycosaminoglycans.

2. The method of claim 1, wherein the hydrolyzed collagen comprises type II collagen.

3. The method of claim 1, wherein the composition comprises at least about 60% protein.

4. The method of claim 1, wherein the composition further comprises at least about 15% chondroitin sulfate.

5. The method of claim 1, wherein the composition further comprises at least about 5% hyaluronic acid.

6. The method of claim 1, wherein the composition comprises at least 60% hydrolyzed type II collagen, 20% chondroitin sulfate, and 10% hyaluronic acid.

7. The method of claim 1, wherein the composition is a hydrolyzed chicken sternal cartilage extract.

8. The method of claim 1, wherein the cell is selected from the group consisting of: an epithelial cell, epidermal cell, myocardial cell, bone marrow cell, B lymphocyte, T lymphocyte, embryonic stem cell, leukocyte, fibroblast, chondrocyte, and osteoblast.

9. The method of claim 8, wherein the cell is a fibroblast.

10. The method of claim 1, wherein the cell is from a subject suffering from a condition associated with reduced telomere length.

11. The method of claim 1, wherein the composition is at a concentration of 25-1000 μg/mL.

12. The method of claim 1, wherein the composition is contained in or combined with a cell culture medium.

13. A method of treating or preventing a condition associated with reduced telomere length, comprising administering a composition to a subject in need thereof, the composition comprising hydrolyzed collagen having an average molecular weight of between about 50 and about 10,000 Da and at least about 20% glycosaminoglycans.

14. The method of claim 13, wherein the hydrolyzed collagen comprises type II collagen.

15. The method of claim 13, wherein the composition comprises at least about 60% protein.

16. The method of claim 13, wherein the composition further comprises at least about 15% chondroitin sulfate.

17. The method of claim 13, wherein the composition further comprises at least about 5% hyaluronic acid.

18. The method of claim 13, wherein the composition comprises at least 60% hydrolyzed type II collagen, 20% chondroitin sulfate, and 10% hyaluronic acid.

19. The method of claim 13, wherein the composition is a chicken sternal cartilage extract.

20. The method of claim 13, wherein the composition is an oral formulation.

21. The method of claim 13, wherein the condition is selected from the group consisting of: bone marrow failure; hematopoietic dysfunction; aplastic anemia (which may be acquired); lung fibrosis; liver fibrosis; cancer; inflammatory bowel disease; ulcerative colitis; Barrett's esophagus; cardiovascular disease; a degenerative disease associated with aging; a telomere syndrome, dyskeratosis congenital; Hoyeraal-Hreidarsson syndrome; Revesz syndrome; cerebroretinal microangiopathy; Fanconi anemia; cryptogenic cirrhosis; nodular regenerative hyperplasia; Rothmund-Thomson syndrome; Immunodeficiency, centromeric region instability, and facial anomalies type I; and, fascioscapulohumeral muscular dystrophy.

22. The method of claim 21, wherein the cancer is colorectal, esophageal, a hematologic cancer, head and neck squamous-cell carcinoma, skin or anorectal cancer, myelodysplastic syndrome, leukemia, or acute myeloid leukemia.

23. A method of promoting at least one of healthy aging, longevity, and wellness, comprising administering a composition to a subject in need thereof, the composition comprising hydrolyzed collagen having an average molecular weight of between about 50 and about 10,000 Da and at least about 20% glycosaminoglycans.

24.-30. (canceled)
